(19) United States
(12) Patent Application Publication
(10) Pub. No.: US 2022/0323310 A1
(43) Pub. Date: Oct. 13, 2022

(54) WIPE AND METHOD FOR REMOVING ODOR FROM A SURFACE, AND METHOD FOR REMOVING STICKINESS FROM A SURFACE

(71) Applicant: LIK LLC, Encino, CA (US)

(72) Inventor: Bryan Bekhrad, Encino, CA (US)

(21) Appl. No.: 17/228,809

(22) Filed: Apr. 13, 2021

(51) Int. Cl. A61K 8/02; A61Q 19/10; A61K 8/60; A61K 8/362; A61K 8/9783; A61K 8/9755

(52) U.S. Cl. CPC A61K 8/0208; A61Q 19/10; A61K 8/602; A61K 8/362; A61K 8/9783; A61K 8/9755

(57) ABSTRACT

A wipe and method for removing odor from a surface, and method for removing stickiness from a surface are presented. The present disclosure includes, but is not limited to, wet wipes and absorbent articles that are capable of cleaning, removing odor, removing stickiness from a surface when used in the intended fashion. More specifically, and without limitation, the present disclosure includes a unique combination of specific amounts of natural occurring ingredients, and/or agents, such as citric acid which aid in the functionality presented herein. The compounds used may provide a surface antimicrobial efficacy during the debris and/or odor removal process. The present disclosure provides a wipe and method of cleaning a surface through removal of odor and/or debris and/or stickiness.

### FIELD OF THE DISCLOSURE

This disclosure relates to a wipe and method for removing odor from a surface, and method for removing stickiness from a surface. More specifically, and without limitation, the present disclosure relates to a wipe for removing odor from the hand of a user. More specifically, and without limitation, the present disclosure relates to cleaning surfaces of an irregular surface and a wipe for doing so. More specifically, and without limitation, the present disclosure relates to cleaning a surface of an irregular surface where the target surface contains an odor and/or stickiness from debris.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright. Bryan Bekhrad. All rights reserved.

### BACKGROUND OF THE DISCLOSURE

The present disclosure relates to a wipe and method for removing odor from a surface, and method for removing stickiness from a surface. More specifically, and without limitation, the present disclosure relates to a wipe for removing odor and/or stickiness and/or debris from the hand or hands of a user.

Various activities are known to cause debris to come into contact with the hands of a user. Hands are known as a humans primary tools and tend to be the primary interaction and/or engagements through which a user interacts with the world around them. Contaminants from soil in gardening to contaminants selected around a household or business to ashes collected on a hand during a smoking activity. Furthermore, odors can collect on hands during activities such as petting an animal, cleaning, smoking, preparing for smoking, a combination thereof, and the like.

Furthermore, even without engaging in any activities, odors, bacteria, debris, and the like accumulate on the skin throughout the course of an ordinary day. These odors and/or debris are often unwanted, causing bad odors or unhealthy potential side effects from spread to the eyes and face, germ spreading to the eyes and face, odor spread, debris spread, and the like.

In the example of a smoking activity, and/or preparing for a smoking activity, a user can accumulate undesired odors and debris on the hands. In one example, when a user engages in the rolling and/or smoking of tobacco causes tobacco debris and tobacco odors to get on the hand. In other words, tobacco debris and odor gets on the hands during the rolling and/or creation process. Similarly, tobacco ash and smoke causes staining of the hands from ash and vapors leaking onto the hands and/or face of a user.

Similarly, in the example of a vaping activity, and/or preparing for a vaping activity, a user can accumulate undesired odors and debris on the hands. In one example, when a user engages in the vaping and/or preparation of vaping, the activity causes vaping debris and/or vaping odors to get on the hand. In other words, vaping debris and odor gets on the hands during the creation and/or preparation process. Similarly, vaping vapor and smoke causes staining of the hands from ash and vapors leaking onto the hands and/or face of a user. This is undesirable as the vapor, ashes, and/or debris can cause staining on the hands of a user, on other surfaces. This is also undesirable because debris, ashes, and vapor can cause odor on the hands, mouth, and face of the user, as well as on other surfaces. Similarly, this is undesirable because the debris, ashes, and vapor from vaping use can cause stickiness on the hands, mouth, and face of the user, as well as on other surfaces.

Similarly, in the example of a smoking activity, and/or preparing for a smoking activity, a user can accumulate undesired odors and debris on the hands. In one example, when a user engages in the rolling and/or smoking of marijuana, the activity causes marijuana debris and marijuana odors to get on the hand. In other words, marijuana debris and odor gets on the hands during the rolling nd/or creation process. Similarly, marijuana ash and smoke causes staining of the hands from ash and vapors leaking onto the hands and/or face of a user. This is undesirable as the vapor, ashes, and/or debris can cause staining on the hands of a user, on other surfaces. This is also undesirable because debris, ashes, and vapor can cause odor on the hands, mouth, and face of the user, as well as on other surfaces. Similarly, this is undesirable because the debris, ashes, and vapor from marijuana use can cause stickiness on the hands, mouth, and face of the user, as well as on other surfaces.

Tobacco stains, vaping stains, stains caused from the use of marijuana are very difficult to remove. Some existing methods include scrubbing with a toothbrush using hydrogen peroxide, and the like. Typical hand washing techniques tend to prove ineffective against this sort of staining. These various removal methods are ineffective against removing the contaminants. This is problematic because these contaminants can be irritating.

In addition to causing odor for a user and/or debris staining for a user and/or stickiness on surfaces (such as hands), the debris from these activities is associated with a number of various ailments. These ailments included, but are not limited to, skin irritation, skin rashes, bacteria growth, bacteria infections, rashes, malodors, as well as other irritant expansion and irritations and appearances that can have various health effects and impacts. Furthermore, in this way, the skin or other surface is not able to function as it typically does through an equilibrium of cell operation, or stickiness, and the like. This can lead to disease, skin cancer, and the more. Furthermore, this can cause healthy bacteria, which are needed on the surface of the skin, to die off and/or disappear around the contaminated area. So for this reason, among others, an easy to use and easy to access means of clearing debris, stains, and odors from the skin is needed in the state of the art.

To date, skin cleaning and/or surface cleaning products, such as water, soap and water, wet wipes, hand towels, sprays, sanitizers, and the like have primarily cleaned the skin by attempting to remove and/or kill all bacteria and/or wash debris from the surface by collecting debris within water and letting the water or other moisture flow from the skin. These methods have been ineffective due to the stickiness of activities, specifically smoking and/or vaping activities and the nature of the debris associated therewith.

Thus, there is a long-felt need in the art for a product and method of use for selectively cleaning and clearing these contaminants from the skin and/or other surface which cause odor, irritations, stickiness, debris concentration, removal of effectively healthy bacteria, and potentially disease, staining, and the like. Furthermore, there is a need in the art for the ability to remove debris, harmful bacteria, stains, and stickiness without the introduction of harmful alcohols which can damage, dry out, and harm skin through reduction of healthy bacteria and the like. Similarly, the existing sanitizers and wipes available fail to solve removal of stickiness and odors commonly caused from smoking and similar activities. Taken in combination there has been a long felt need in the art for a healthy and effective product and method of use to achieve these needs.

The disclosure herein provides these advantages to the state of the art, and others, as will become clear from the specification and claims provided.

### SUMMARY OF THE DISCLOSURE

A wipe and method for removing odor from a surface, and method for removing stickiness from a surface are presented. More specifically, and without limitation, the present disclosure relates to a wipe for removing odor from the hand of a user. More specifically, and without limitation, the present disclosure relates to cleaning surfaces of an irregular surface and a wipe for doing so. More specifically, and without limitation, the present disclosure relates to cleaning a surface of an irregular surface where the target surface contains an odor and/or stickiness from debris.

In the present disclosure, as one example, the present disclosure is directed to a hand wipe and method of use for quickly, efficiently, and effectively clearing debris from the hands of a user which has accumulated as a result of a smoking and/or marijuana and/or vaping related activity. Said another, the present disclosure provides the state of the art with a cannabis odor and resin (single hand) wipe. Said another way, the present disclosure is designed and configured to remove stickiness, as caused by cannabis resin, from the hands of a user while also removing the odor of cannabis from the hands of a user (including the removal of cannabis ash from the hands of a user).

Thus, the present disclosure provides these advantages and more as novel features, among other portions of this disclosure provide huge advantages and improvements to traditional surface cleaning wipes and methods. For this reason, and in similar circumstances, the present disclosure provides unique solutions and novel functionality.

Thus, it is a primary object of the disclosure to provide a wipe and method for removing odor from a surface, and method for removing stickiness from a surface that provides novel functionality and improves upon the state of the art of surface cleaning.

Another object of the disclosure is to provide a wipe and method for removing odor from a surface, and a method for removing stickiness from a surface that provides for cannabis odor cleaning.

Yet another object of the disclosure is to provide a wipe and method for removing odor from a surface, and a method for removing stickiness from a surface that provides a cannabis resin removal wipe.

Another object of the disclosure is to provide a wipe and method for removing odor from a surface, and a method for removing stickiness from a surface that provides a cannabis odor removal wipe.

Yet another object of the disclosure is to provide a wipe and method for removing odor from a surface, and a method for removing stickiness from a surface that provides for hand cleaning of odor and resins.

Another object of the disclosure is to provide a wipe and method for removing odor from a surface, and a method for removing stickiness from a surface that provides irregular surface cleaning.

Yet another object of the disclosure is to provide a wipe and method for removing odor from a surface, and a method for removing stickiness from a surface that is environmentally friendly.

Another object of the disclosure is to provide a wipe and method for removing odor from a surface, and a method for removing stickiness from a surface that utilizes only naturally occurring ingredients.

Yet another object of the disclosure is to provide a wipe and method for removing odor from a surface, and a method for removing stickiness from a surface that reduces water waste.

Another object of the disclosure is to provide a wipe and method for removing odor from a surface, and a method for removing stickiness from a surface that provides for isolated cleaning of a surface.

Yet another object of the disclosure is to provide a wipe and method for removing odor from a surface, and a method for removing stickiness from a surface that provides for cleaning delicate surfaces.

Another object of the disclosure is to provide a wipe and method for removing odor from a surface, and a method for removing stickiness from a surface that provides time savings for a user.

Yet another object of the disclosure is to provide a wipe and method for removing odor from a surface, and a method for removing stickiness from a surface that provides convenience for a user.

Another object of the disclosure is to provide a wipe and method for removing odor from a surface, and a method for removing stickiness from a surface that provides a fully contained system.

Yet another object of the disclosure is to provide a wipe and method for removing odor from a surface, and a method for removing stickiness from a surface that recovers fluids applied to a surface.

Another object of the disclosure is to provide a wipe and method for removing odor from a surface, and a method for removing stickiness from a surface that provides ease of use for a user.

Yet another object of the disclosure is to provide a wipe and method for removing odor from a surface, and a method for removing stickiness from a surface that removes stickiness of cannabis resin from the hands of a user.

Another object of the disclosure is to provide a wipe and method for removing odor from a surface, and a method for removing stickiness from a surface that removes odor caused by cannabis from the hands of a user.

Another object of the disclosure is to provide a wipe and method for removing odor from a surface, and a method for removing stickiness from a surface that does not implement harmful alcohols.

Yet another object of the disclosure is to provide a wipe and method for removing odor from a surface, and a method for removing stickiness from a surface that utilizes citric acid.

Another object of the disclosure is to provide a wipe and method for removing odor from a surface, and a method for removing stickiness from a surface that provides for a pleasant scent.

Yet another object of the disclosure is to provide a wipe and method for removing odor from a surface, and a method for removing stickiness from a surface that is easy to use.

Another object of the disclosure is to provide a wipe and method for removing odor from a surface, and a method for removing stickiness from a surface that is safe to use.

Yet another object of the disclosure is to provide a wipe and method for removing odor from a surface, and a method for removing stickiness from a surface that can be applied in an isolated area.

Another object of the disclosure is to provide a wipe and method for removing odor from a surface, and a method for removing stickiness from a surface that can be odor free.

Yet another object of the disclosure is to provide a wipe and method for removing odor from a surface, and a method for removing stickiness from a surface that is easy to dispose of.

Another object of the disclosure is to provide a wipe and method for removing odor from a surface, and a method for removing stickiness from a surface that is quick.

Yet another object of the disclosure is to provide a wipe and method for removing odor from a surface, and a method for removing stickiness from a surface that is efficient.

Another object of the disclosure is to provide a wipe and method for removing odor from a surface, and a method for removing stickiness from a surface that is high quality.

Yet another object of the disclosure is to provide a wipe and method for removing odor from a surface, and a method for removing stickiness from a surface that can dispense and dispose with ease.

Another object of the disclosure is to provide a wipe and method for removing odor from a surface, and a method for removing stickiness from a surface that can dispense easily even when hands are contaminated.

Yet another object of the disclosure is to provide a wipe and method for removing odor from a surface, and a method for removing stickiness from a surface that minimizes environmental impact through packaging and the like.

Another object of the disclosure is to provide a wipe and method for removing odor from a surface, and a method for removing stickiness from a surface that improves skin health.

Yet another object of the disclosure is to provide a wipe and method for removing odor from a surface, and a method for removing stickiness from a surface that provides an all in one article for cleansing and absorption.

Another object of the disclosure is to provide a wipe and method for removing odor from a surface, and a method for removing stickiness from a surface that promotes healthy skin.

Yet another object of the disclosure is to provide a wipe and method for removing odor from a surface, and a method for removing stickiness from a surface that inhibits the growth of harmful bacteria, viruses, and the like.

Another object of the disclosure is to provide a wipe and method for removing odor from a surface, and a method for removing stickiness from a surface that removes harmful contaminants.

Yet another object of the disclosure is to provide a wipe and method for removing odor from a surface, and a method for removing stickiness from a surface that removes odors rather than masks odors.

Another object of the disclosure is to provide a wipe and method for removing odor from a surface, and a method for removing stickiness from a surface that inhibits the adherence of harmful substances to the surface of the skin or other surfaces.

Yet another object of the disclosure is to provide a wipe and method for removing odor from a surface, and a method for removing stickiness from a surface that prevents yeast growth.

Another object of the disclosure is to provide a wipe and method for removing odor from a surface, and a method for removing stickiness from a surface that provides an impregnated article.

Yet another object of the disclosure is to provide a wipe and method for removing odor from a surface, and a method for removing stickiness from a surface that provides a comfortable fibrous material for use as a wipe.

Another object of the disclosure is to provide a wipe and method for removing odor from a surface, and a method for removing stickiness from a surface that cleans dirt and oil from a surface.

Yet another object of the disclosure is to provide a wipe and method for removing odor from a surface, and a method for removing stickiness from a surface that cleans dirt and oil and odors caused by food from a surface.

These and other objects, features, or advantages of the present disclosure will become apparent from the specification and claims.

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

The present disclosure relates to products and methods for cleaning, removing, and deodorizing a surface. More specifically, and without limitation, the present disclosure relates to products, such as wet wipes or absorbent articles containing one or more compounds that promote cleaning, removal of contaminants, and deodorizing of a surface and inhibit the growth of bacteria and other microorganisms, or any combination thereof.

In one embodiment, the present disclosure is directed to promoting cleaning, removing, and deodorizing a surface. The product comprises a unique substrate composition that is capable of effectively removing stickiness from a surface, removing odors from a surface, as further described herein.

In another embodiment, the present disclosure is directed to wipes or absorbent articles, for inhibiting growth of odor causing bacteria, removing debris and/or stickiness from a surface, as further described herein. The wipe or absorbent material includes a substrate with a high amount of citric acid compared to other cleaning agents. The present disclosure also relates to a citric acid concentration of a substrate or an absorbent substrate of 12.0 weight percent or higher, as defined herein.

In another embodiment, the present disclosure is directed to a product and/or composition, for inhibiting growth of odor causing bacteria, removing debris and/or stickiness from a surface, as further described herein. The wipe or absorbent material includes a substrate with a high amount of citric acid compared to other cleaning agents. The present disclosure also relates to a citric acid concentration of a substrate or an absorbent substrate of 12.0 weight percent or higher, as defined herein.

The present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water and at least a second compound such as citric acid.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and at least a third compound such as eucalyptus globulus and/or eucalyptus globulus essential oil, or similar compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and at least a third compound such as Mentha piperita and/or Mentha piperita essential oil, or similar compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and at least a third compound such as decyl glucoside, or similar compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and a third compound such as eucalyptus globulus, and at least a fourth compound such as Mentha piperita essential oil, or similar compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and a third compound such as eucalyptus globulus, and at least a fourth compound such as decyl glucoside, or similar compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and a third compound such as eucalyptus globulus, and a fourth compound such as decyl glucoside, and at least a fifth compound such as Mentha piperita and/or Mentha piperita essential oil, or similar compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and a third compound such as eucalyptus globulus, and a fourth compound such as decyl glucoside, and at least a fifth compound such as Mentha piperita and/or Mentha piperita essential oil, and at least a sixth compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and a third compound such as eucalyptus globulus, and a fourth compound such as decyl glucoside, and at least a fifth compound such as Mentha piperita and/or Mentha piperita essential oil, and at least a sixth compound, and at least a seventh compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and a third compound such as eucalyptus globulus, and a fourth compound such as decyl glucoside, and at least a fifth compound such as Mentha piperita and/or Mentha piperita essential oil, and at least a sixth compound, and at least a seventh compound, and at least an eighth compound.

In another embodiment, the present disclosure is directed to a substrate and/or composition of a substrate that may be a fibrous or absorbent material and/or absorbent substrate, and a combination of two or more compounds that may provide differing beneficial effects. In this embodiment, the product comprises a first compound such as water, and a second compound such as citric acid, and a third compound such as eucalyptus globulus, and a fourth compound such as decyl glucoside, and at least a fifth compound such as Mentha piperita and/or Mentha piperita essential oil, and at least a sixth compound, and at least a seventh compound, and at least an eighth compound, and at least a ninth compound, or more compounds.

While these compounds are hereby contemplated for use a various combinations and quantities, a number of similar compounds or adequate replacements of the various compounds contemplated herein are also hereby contemplated for use.

In this way, the present disclosure relates to products and methods of use that remove debris, remove stickiness, clean a surface, remove odors, prevent bacteria growth and/or prevent the growth of microorganisms, provide pleasant scents to a surface, provide comfort and cleanliness, and promote improved surface comfort, coat a surface in a compound which prevents future bacterial growth and future odors, and the like.

Wipe and/or Product:

In accordance with the present disclosure, it has been discovered that certain compounds, combined in a unique way, provide for and can be utilized with personal care products, such as wipes, hand wipes, surface wipes, in a spray bottle, or with absorbent materials, such as absorbent articles, to apply to a skin surface or other surface various benefits from cleanliness to debris removal, to stickiness removal, to odor removal, to provide improved and/or desirable scents, and provide antimicrobial benefits to the skin or another surface.

More specifically, and without limitation, the present disclosure provides a unique combination of compounds and a wipe and/or product that provides at least one of the following benefits when applied to the surface of the skin and/or applied to a surface: (1) promotes effective removal of debris and particularly smoke, ash, or debris related to smoking, food, and/or drink; (2) removes and/or kills odor causing bacteria and/or odor causing agents that tend to stick to the skin, such as a hand, a face, and the like and/or other surfaces such as a barbecue grill, cooking surface, and the like; (3) inhibits and/or removes and/or kills bacterial growth and/or microbial growth and/or odor causing organisms and the like.

In accordance with the present disclosure, the compound and/or solution created for a substrate is highly suitable for a spray application and/or in a wipe application and/or in an absorbent material application. Furthermore, and in this way, the solution that is used in combination with an absorbent article substrate, a user can control the contents of stickiness, tobacco and cannabis debris, and remove unwanted odors, while replacing with desirable scents and desirable organic, safe materials which are safe for application to skin and other surfaces, while also providing bacteria and yeast control for a surface.

In one embodiment, the present disclosure is directed toward personal care products and particularly a compound, and is meant for use on human skin such as hands, arms, face, neck, legs, and other skin surfaces. The compound or unique combination of contents and creation of a unique personal care product which improve the health, smell, feel, and the like of a skin surface and particularly hands. Once the surface is contacted by a wipe or absorbent article, the wipe with substrate and/or the absorbent article, in the present disclosure impart health improvements and the like for a user.

Furthermore, in this embodiment, the combination of essential oils and the like within the compound, of the present disclosure, are combined with wipes, such as wet wipes or hand wipes, or cosmetic wipes, or face wipes, or cleaning wipes, or surface wipes, or feminine wipes, or cooking wipes, and the like to remove debris, remove stickiness, remove odor and control the growth of bacteria and/or control the growth of microorganisms, and even promote the ability of the growth of healthy bacteria.

In the present embodiment, the substrate and/or compound provided in the present disclosure is provided with a particular type of wipe. However, the application of this compound is not limited to a wipe and may be applied to other materials. Examples of other materials include, but are not limited to clothing, socks, shoes, gloves, diapers, baby wipes, training pants, adult incontinence garments, napkins, tissue, wound cleaning products, paper towels, and other materials suitable for accepting a substrate. Other materials may be materials such as absorbent materials, fibrous materials, and the like. In this way, the present disclosure provides a compound which is mostly liquid and can be impregnated on a wipe or other material.

Furthermore, although the above describes the present disclosure as a compound which is impregnated with or upon an absorbent material or the like, the present disclosure can also be used with various other materials in other ways. Some examples of a incorporation of the compound of the present disclosure may include applications to fibrous sheets, non-woven fibrous sheets, and the like. Similarly, for applications other than impregnated fluids, the present disclosure may include incorporation means including, but not limited to, integrating the compound directly into the construction process of materials such as weaving, conforming, air-laid, hydroentangling, and other combinations of interweaving with fibers and into polymers and molding, and the like.

In another embodiment, the present disclosure is directed toward personal care products and particularly a compound, and is formed of an ointment to be applied to a surface, the ointment to be squeezed from a bottle or the like. In this way, the present disclosure, and in this embodiment, is meant for and can be applied to use on human skin such as hands, arms, face, neck, legs, and other skin surfaces. In this way, this embodiment would be rubbed into or applied to skin, typically by use of a user's hands (as one common application). The compound or unique combination of contents and creation of a unique personal care product which improves the health, smell, feel, and the like of a skin surface and particularly hands. Once the surface is contacted by a wipe or absorbent article, the wipe with substrate and/or the absorbent article, in the present disclosure, impart health improvements and the like for a user.

In another embodiment, the present disclosure is directed toward personal care products and particularly a compound, and is formed of a spray to be applied to a surface, the spray could be ejected from a bottle and/or pressurized bottle and can even be mixed with air upon ejection and/or mixed with pressurized air and the like. In this way, the present disclosure, and in this embodiment, is meant for and can be applied to use on human skin such as hands, arms, face, neck, legs, and other skin surfaces. In this way, this embodiment would be rubbed into or applied to skin, typically by use of a user's hands (as one common application). The compound or unique combination of contents and creation of a unique personal care product which improves the health, smell, feel, and the like of a skin surface and particularly hands. Once the surface is contacted by a wipe or absorbent article, the wipe with substrate and/or the absorbent article, in the present disclosure, impart health improvements and the like for a user.

In the present disclosure, and as mentioned herein, one embodiment is directed toward a wipe suitable for use in combination with the compound herein to improve upon skin health and the like, which in addition to the wipe or material as mentioned herein, also includes a liquid solution or formulation.

In the present disclosure, the liquid solution or formulation can be any liquid, any liquid which can be absorbed into a wet wipe or similar material, and may include various suitable components. For example, and in one embodiment of the present disclosure, the components may include water, solubles, creams, lotions, medicaments, antimicrobials, antibiotics, and other emollients, surfactants, scents or fragrances, agents, buffers, or any combination thereof.

The formulations in various applications of the present disclosure may vary depending on the application of the compound. For example, the liquid amount within each wet wipe may vary depending on the material type being used to provide the wet wipe. Furthermore, the liquid amount within each wet wipe may also vary depending on the packaging being used to provide the wet wipe. Furthermore, the liquid amount within each wet wipe may also vary depending on desired end use of the wet wipe.

In one embodiment herein, a small single use wet wipe is provided from a sealed package, both of which are discarded after use. In this embodiment, as one example, the wet wipe and packaging can be likened to wet wipes and general wetness as is known in the art for hand wipes. In this way, and in this embodiment, generally, each wet wipe is rectangular or square in shape and may be folded within the packaging. A package may include one wet wipe for a single hand, or may include two wet wipes, one for each hand, in this type of single use application.

In another embodiment, wet wipes are provided for various surface cleaning, such as in and around food preparation areas. In this embodiment, the wet wipes may be provided as a plurality of wet wipes which are stored within a disposable container likely housing many wet wipes that are used and applied over a period of time. In this way, and in this embodiment, the wet wipes may be generally rectangular or square and may be in a rolled format such that a single wet wipe can be unrolled and/or dispensed for use. Similarly, the wet wipes, as a roll may be a single material with perforations such that each wipe tears during the dispensing process. These arrangements and other applications are hereby contemplated for use.

In one specific embodiment, the present disclosure is formed of a compound absorbed by a wet wipe which includes the formulation comprising: 86.10 weight percent water, 12.00 weight percent citric acid, 0.40 weight percent of decyl glucoside (or 0.20 weight percent decyl glucoside and 0.20 weight percent sodium lauryl sulfate, or equivalent), 1.25 weight percent Mentha piperita essential oil, and 0.25 weight percent eucalyptus globulus essential oil. This combination forms an example of a compound as presented herein. These compounds and/or equivalents are hereby contemplated for use.

In another embodiment, the present disclosure is formed of a compound of the formulation comprising: water, at least 12.00 weight percent citric acid, decyl glucoside (or equivalent), Mentha piperita, eucalyptus globulus, other components such as fragrances, and the like. Various combinations of these ingredients and, the like, are hereby contemplated for use an embodiment herein.

In another embodiment, the present disclosure is formed of a compound of the formulation comprising: 80-90 weight percent water, 10-20 weight percent citric acid, 0.01-1.00 weight percent decyl glucoside (or equivalent), 0.01-2.00 weight percent Mentha piperita, 0.01-2.00 weight percent eucalyptus globulus, and other ingredients. Various combinations of these ingredients, and the like, are hereby contemplated for use as an embodiment herein. These combinations and ranges may vary slightly as applicable to various applications and various methods of use.

In another embodiment, the present disclosure is formed of a compound of the formulation comprising: 70-95 weight percent water, 5-30 weight percent citric acid, 0.01-2.00 weight percent decyl glucoside (or equivalent), 0.01-5.00 weight percent Mentha piperita, 0.01-5.00 weight percent eucalyptus globulus, and other ingredients. Various combinations of these ingredients, and the like, are hereby contemplated for use as an embodiment herein. These combinations and ranges may vary slightly as applicable to various applications and various methods of use.

In another embodiment, the present disclosure is formed of a compound of the formulation comprising: 60-99 weight percent water, 1-40 weight percent citric acid, 0.01-5.00 weight percent decyl glucoside (or equivalent), 0.01-8.00 weight percent Mentha piperita, 0.01-8.00 weight percent eucalyptus globulus, and other ingredients. Various combinations of these ingredients, and the like, are hereby contemplated for use as an embodiment herein. These combinations and ranges may vary slightly as applicable to various applications and various methods of use.

In another embodiment, the present disclosure is formed of a compound of the formulation comprising: 50-99.9 weight percent water, 0.1-50 weight percent citric acid, 0.01-10.00 weight percent decyl glucoside (or equivalent), 0.01-10.00 weight percent Mentha piperita, 0.01-10.00 weight percent eucalyptus globulus, and other ingredients. Various combinations of these ingredients, and the like, are hereby contemplated for use as an embodiment herein. These combinations and ranges may vary slightly as applicable to various applications and various methods of use.

In another embodiment, the present disclosure is formed of a compound of the formulation comprising: 40-99.9 weight percent water, 0.1-80 weight percent citric acid, 0.01-15.00 weight percent decyl glucoside (or equivalent), 0.01-15.00 weight percent Mentha piperita, 0.01-15.00 weight percent eucalyptus globulus, and other ingredients. Various combinations of these ingredients, and the like, are hereby contemplated for use as an embodiment herein. These combinations and ranges may vary slightly as applicable to various applications and various methods of use.

In another embodiment, the present disclosure is formed of a compound of the formulation comprising: 10.0-99.9 weight percent water, 0.1-90.0 weight percent citric acid, 0.01-20.00 weight percent decyl glucoside (or equivalent), 0.01-20.00 weight percent Mentha piperita, 0.01-20.00 weight percent eucalyptus globulus, and other ingredients. Various combinations of these ingredients, and the like, are hereby contemplated for use as an embodiment herein. These combinations and ranges may vary slightly as applicable to various applications and various methods of use.

In another embodiment, the present disclosure is formed of a compound of the formulation comprising: 1.0-99.9 weight percent water, 0.1-99.0 weight percent citric acid, 0.01-20.00 weight percent decyl glucoside (or equivalent), 0.01-20.00 weight percent Mentha piperita, 0.01-20.00 weight percent eucalyptus globulus, and other ingredients. Various combinations of these ingredients, and the like, are hereby contemplated for use as an embodiment herein. These combinations and ranges may vary slightly as applicable to various applications and various methods of use.

In another specific embodiment, the present disclosure is formed of a compound of the formulation comprising: 80.0-90.0 weight percent water, 10.0-20.0 weight percent citric acid, 0.01-5.00 weight percent decyl glucoside (or equivalent), 0.01-5.00 weight percent Mentha piperita, and other ingredients. Various combinations of these ingredients, and the like, are hereby contemplated for use as an embodiment herein. These combinations and ranges may vary slightly as applicable to various applications and various methods of use.

In another specific embodiment, the present disclosure is formed of a compound of the formulation comprising: 80.0-90.0 weight percent water, 10.0-20.0 weight percent citric acid, 0.01-5.00 weight percent decyl glucoside (or equivalent), and other ingredients. Various combinations of these ingredients, and the like, are hereby contemplated for use as an embodiment herein. These combinations and ranges may vary slightly as applicable to various applications and various methods of use.

In another specific embodiment, the present disclosure is formed of a compound of the formulation comprising: 80.0-90.0 weight percent water, 10.0-20.0 weight percent citric acid, and other ingredients. Various combinations of these ingredients, and the like, are hereby contemplated for use as an embodiment herein. These combinations and ranges may vary slightly as applicable to various applications and various methods of use.

In another specific embodiment, the present disclosure is formed of a compound of the formulation comprising: 70.0-90.0 weight percent water, 10.0-30.0 weight percent citric acid, and other ingredients. Various combinations of these ingredients, and the like, are hereby contemplated for use as an embodiment herein. These combinations and ranges may vary slightly as applicable to various applications and various methods of use.

The present disclosure is not intended to be limited to one set of ingredients. The present disclosure may also comprise a combination of other compounds and/or ingredients. In this way, the present disclosure may have additional compounds and/or ingredients to provide multiple benefits to an end user. Various other combinations may be utilized and/or incorporated into a wipe and/or absorbent material and/or spray and/or cream for use in combination with the compound disclosed herein. In this way, the present disclosure may provide additional benefits beyond those expressly mentioned herein. For example, one compound may include additional ingredients which provide moisturizing for skin, scents for surfaces, and the like.

In addition to the above identified features, options, combinations, and applications, the product and/or compound may also include other features and functionalities, among other options, compositions, and applications.

Methods of Use:

As one example, the present disclosure is directed toward a method for cleaning debris, removing stickiness, and removing odor from a surface such as skin. The method includes contacting a fibrous wipe or absorbent material with a compound and subsequently contacting the fibrous wipe or absorbent material with the surface of the skin. The compound includes a compound as is disclosed herein, wherein the compound is selected from the compounds discussed herein.

Said another way, and as one example, the present method of use is directed toward personal care products and particularly a compound, and is formed of a spray to be applied to a surface, the spray could be ejected from a bottle and/or pressurized bottle and can even be mixed with air upon ejection and/or mixed with pressurized air and the like. In this way, the present disclosure, and in this embodiment, is meant for and can be applied to use on human skin such as hands, arms, face, neck, legs, and other skin surfaces. In this way, this embodiment would be rubbed into or applied to skin, typically by use of a user's hands (as one common application). The compound or unique combination of contents and creation of a unique personal care product which improves the health, smell, feel, and the like of a skin surface and particularly hands. Once the surface is contacted by a wipe or absorbent article, the wipe with substrate and/or the absorbent article, in the present disclosure, impart health improvements and the like for a user.

As another example, the present method of use is directed toward personal care products and particularly a compound directed toward a wipe suitable for use in combination with the compound herein to improve upon skin health and the like, which in addition to the wipe or material as mentioned herein, also includes a liquid solution or formulation.

As another example, the present method of use is directed toward personal care products and particularly a compound directed toward a liquid solution or formulation can be any liquid, any liquid which can be absorbed into a wet wipe or similar material, and may include various suitable components. For example, and in one embodiment of the present disclosure, the components may include water, solubles, creams, lotions, medicaments, antimicrobials, antibiotics, and other emollients, surfactants, scents or fragrances, agents, buffers, or any combination thereof.

The formulations in various applications of the present disclosure may vary depending on the application of the compound. For example, the liquid amount within each wet wipe may vary depending on the material type being used to provide the wet wipe. Furthermore, the liquid amount within each wet wipe may also vary depending on the packaging being used to provide the wet wipe. Furthermore, the liquid amount within each wet wipe may also vary depending on desired end use of the wet wipe.

As one example, a method of use for cleaning a surface is directed to a small single use, wet wipe provided from a sealed package, both of which are discarded after use. In this embodiment, as one example of a method of use, the wet wipe and packaging can be likened to wet wipes and general wetness as is known in the art for hand wipes. In this way, and in this method of use, generally, each wet wipe is rectangular or square in shape and may be folded within the packaging. A package may include one wet wipe for a single hand, or may include two wet wipes, one for each hand, in this type of single use application.

As one example, a method of use for cleaning a surface is directed to wet wipes for various surface cleaning, such as in and around food preparation areas. In this example of a method of use, the wet wipes may be provided as a plurality of wet wipes which are stored within a disposable container likely housing many wet wipes that are used and applied over a period of time. In this way, and in this method of use, the wet wipes may be generally rectangular or square and may be in a rolled format such that a single wet wipe can be unrolled and/or dispensed for use. Similarly, the wet wipes, as a roll may be a single material with perforations such that each wipe tears during the dispensing process. These arrangements and other applications are hereby contemplated for use.

As one example, a method of use for cleaning a surface is directed to provides a unique combination of compounds and a wipe and/or product that provides at least one of the following benefits when applied to the surface of the skin and/or applied to a surface: (i) promotes effective removal of debris and particularly smoke, ash, or debris related to smoking, food, and/or drink; (ii) removes and/or kills odor causing bacteria and/or odor causing agents that tend to stick to the skin, such as a hand, a face, and the like and/or other surfaces such as a barbecue grill, cooking surface, and the like; (iii) inhibits and/or removes and/or kills bacterial growth and/or microbial growth and/or odor causing organisms and the like.

In accordance with the present method of use, as one example, the compound and/or solution created for a substrate is highly suitable for a spray application and/or in a wipe application and/or in an absorbent material application. Furthermore, and in this way, the solution that is used in combination with an absorbent article substrate, a user can control the contents of stickiness, tobacco and cannabis debris, and remove unwanted odors, while replacing with desirable scents and desirable organic, safe materials which are safe for application to skin and other surfaces, while also providing bacteria and yeast control for a surface.

In another example, as a method of use, the present disclosure is directed toward personal care products and particularly a compound, and is meant for use on human skin such as hands, arms, face, neck, legs, and other skin surfaces. The compound or unique combination of contents and creation of a unique personal care product which improves the health, smell, feel, and the like of a skin surface and particularly hands. Once the surface is contacted by a wipe or absorbent article, the wipe with substrate and/or the absorbent article, in the present disclosure, impart health improvements and the like for a user.

It will be appreciated by those skilled in the art that other various modifications could be made to the products, compounds, and methods of use without parting from the spirit and scope of this disclosure. All such modifications and changes fall within the scope of the claims and are intended to be covered thereby.

What is claimed:

1. A product for cleaning a surface and promoting cleanliness, comprising:
an absorbent article;
a compound comprised of a plurality of substances;
the compound having a first substance capable of removing debris from the surface;
wherein the first substance is made of at least 50 weight percent water;
the compound having a second substance capable of removing odor from a surface;
wherein the second substance is made of at least 5 weight percent citric acid;
wherein the second substance is capable of preventing bacteria growth on a surface.

2. The product for cleaning a surface and promoting cleanliness of claim 1, further comprising:
wherein the compound is a liquid formulation.

3. The product for cleaning a surface and promoting cleanliness of claim 1, further comprising:
wherein the absorbent article is a wipe substrate.

4. The product for cleaning a surface and promoting cleanliness of claim 1, further comprising:
wherein the absorbent article is an absorbent substrate.

5. The product for cleaning a surface and promoting cleanliness of claim 1, further comprising:
wherein the absorbent article is a hand wipe.

6. The product for cleaning a surface and promoting cleanliness of claim 1, further comprising:
the compound having a third substance;
wherein the third substance is decyl glucoside.

7. The product for cleaning a surface and promoting cleanliness of claim 1, further comprising:
the compound having a third substance;
wherein the third substance is decyl glucoside;
the compound having a fourth substance;
wherein the fourth substance is Mentha piperita.

8. The product for cleaning a surface and promoting cleanliness of claim 1, further comprising:
the compound having a third substance;
wherein the third substance is decyl glucoside;
the compound having a fourth substance;
wherein the fourth substance is Mentha piperita;
the compound having a fifth substance;
wherein the fifth substance is eucalyptus globulus.

9. The product for cleaning a surface and promoting cleanliness of claim 1, further comprising:
wherein the surface is the skin of a human.

10. The product for cleaning a surface and promoting cleanliness of claim 1, further comprising:
wherein the surface is the skin of a human;
wherein the product for cleaning a surface and promoting cleanliness is removing odor caused by smoking activities.

11. The product for cleaning a surface and promoting cleanliness of claim 1, further comprising:
wherein the surface is the skin of a human;
wherein the product for cleaning a surface and promoting cleanliness is removing odor caused by smoking activities;
wherein the smoking activities are cannabis use.

12. The product for cleaning a surface and promoting cleanliness of claim 1, further comprising:
wherein the surface is the skin of a human;
wherein the product for cleaning a surface and promoting cleanliness is removing stickiness caused by smoking activities;
wherein the smoking activities are cannabis use.

13. The product for cleaning a surface and promoting cleanliness of claim 1, further comprising:
wherein the compound comprises:
a first substance being present on the absorbent article in an amount of 86.1 weight percent of the compound;
a second substance being present on the absorbent article in an amount of 12.0 weight percent of the compound;
a third substance being present on the absorbent article in an amount of 0.40 weight percent of the compound;
a fourth substance being present on the absorbent article in an amount of 1.25 weight percent of the compound;
a fifth substance being present on the absorbent article in an amount of 0.25 weight percent of the compound.

14. A product for promoting odor removal from a surface, the product comprising:
an absorbent article;
the absorbent article being saturated with a compound;
wherein a first substance is present in the compound capable of removing debris from the surface;
wherein the first substance present in the compound is about 80 to 85 percent water (80 to 85 weight percent water of the compound);
wherein a second substance is present in the compound capable of removing odor from a surface;
wherein the second substance present in the compound is about 10 to 20 percent citric acid (10 to 20 weight percent citric acid of the compound);
wherein the second substance is capable of preventing bacteria growth on a surface.

15. The product for cleaning a surface and promoting cleanliness of claim 14, further comprising:
wherein the compound is a liquid formulation.

16. The product for cleaning a surface and promoting cleanliness of claim 14, further comprising:
wherein the absorbent article is a wipe substrate.

17. The product for cleaning a surface and promoting cleanliness of claim 14, further comprising:
wherein the absorbent article is an absorbent substrate.

18. The product for cleaning a surface and promoting cleanliness of claim 14, further comprising:
wherein the compound comprises:
wherein the third substance present in the compound is about 0.01 to 1.5 percent decyl glucoside (0.01 to 1.5 weight percent decyl glucoside of the compound);
wherein the fourth substance present in the compound is about 0.01 to 2.5 percent Mentha piperita (0.01 to 2.5 weight percent Mentha piperita of the compound);
wherein the fifth substance present in the compound is about 0.01 to 1.5 percent eucalyptus globulus (0.01 to 1.5 weight percent eucalyptus globulus of the compound).

19. A method of cleaning a surface using a product for cleaning a surface, the steps comprising:
providing an absorbent article;
providing a compound; wherein the compound comprises a first substance and a second substance capable of removing debris; wherein the first substance comprises about 80 weight percent water; wherein the second substance comprises about 12 weight percent citric acid;
impregnating the absorbent article with the compound;
wiping a surface with the absorbent article.

20. A method of claim 19, further comprising the steps:
packaging the absorbent article;
removing the absorbent article from the packaging;
impregnating the absorbent article with a third substance comprising about 1 weight percent of the compound;
impregnating the absorbent article with a fourth substance comprising about 1 weight percent of the compound;
impregnating the absorbent article with a fifth substance comprising about 1 weight percent of the compound.
